(19) United States
(12) Patent Application Publication
(10) Pub. No.: US 2025/0099590 A1
(43) Pub. Date: Mar. 27, 2025

(54) HIGH DENSITY ULTRASOUND COUPLING GEL AND METHODS OF USE THEREOF

(71) Applicant: ZetrOZ Systems LLC, Trumbull, CT (US)

(72) Inventor: George K. LEWIS, JR., Trumbull, CT (US)

(73) Assignee: ZetrOZ Systems LLC, Trumbull, CT (US)

(21) Appl. No.: 18/439,728

(22) Filed: Feb. 12, 2024

Related U.S. Application Data
(60) Provisional application No. 63/444,878, filed on Feb. 10, 2023.

(51) Int. Cl. A61K 41/00; A61K 31/196; A61N 7/02

(52) U.S. Cl. CPC A61K 41/0047; A61K 31/196; A61N 7/02

(57) ABSTRACT

The present disclosure relates to a high density ultrasound coupling gel having improved ultrasound coupling, thermal transfer, drug delivery, and long duration ultrasound treatment. The high density ultrasound coupling gel includes a water-based ultrasound transmission gel material in a concentration amount of between about 90 and 99 weight percent (wt %) and a sodium dopant in a concentration amount of between about 1 and 10 wt %. The water-soluble ultrasound transmission gel material is effective to conduct acoustic energy. The sodium dopant is effective to increase density of the water-based ultrasound transmission gel material. The high density ultrasound coupling gel has a density of between about 1 and 2 grams per milliliter (g/mL). The present disclosure also relates to the use of the high density ultrasound coupling gel in ultrasound coupling devices, ultrasound systems, and methods of treatment such as long-duration ultrasound therapeutic treatment.

### CROSS REFERENCE TO RELATED APPLICATIONS

This application claims priority benefit of U.S. Provisional Patent Application Ser. No. 62/444,878, filed Feb. 10, 2023, the disclosure of which is hereby incorporated by reference herein in its entirety.

### FIELD OF THE INVENTION

The present disclosure generally relates to, inter alia, a high density ultrasound coupling gel, ultrasound coupling device (e.g., coupling gel patch), and ultrasound system for improved ultrasound coupling, thermal transfer, drug delivery, and long duration ultrasound treatment.

### BACKGROUND

Low-intensity therapeutic ultrasound (LITUS) therapy is increasingly used to heal soft tissue injuries and relieve pain in acute and chronic musculoskeletal ailments [1-4]. Ultrasound penetrates the damaged tissue to enhance the healing process by several mechanisms, including diathermy and mechano-transduction [5-8]. The localized increase in the temperature, cellular migration, and biomolecular activity enhances circulation, vasodilation, oxygenation, and nutrient exchange, boosting cellular metabolism and tissue regeneration [6-11].

The LITUS is transmitted at frequencies between 1 and 3 MHz at intensities of less than 0.5 W/cm² for 10 minutes to 4 hours [4, 12]. The consistent and soothing vigorous heat penetrates deep into connective tissue and muscles with little to no adverse effects on the tissue [9, 13]. The application of low-intensity ultrasound between 2-20 MHz is also used in clinical diagnostic and imaging purposes, depending on the application and target tissue. The diagnostic and imaging use of ultrasound rely on the difference in acoustic impedance of tissues. The use of high-intensity focused ultrasound (HIFUS) uses >10 W/cm² intensity, generating localized heating, more commonly applied in targeting solid tumors. The HIFUS can be used as an alternative to invasive surgery [14].

Diclofenac Sodium is a broadly used non-steroidal anti-inflammatory drug (NSAID). It is an analgesic, anti-inflammatory, and antipyretic agent [15]. It acts by inhibiting cyclooxygenase-1 and 2, regulating the production of prostaglandin and various pro-inflammatory biomarkers, including tumor factor alpha (TNFα), interleukin-6 (IL-6), and interleukin-8 (IL-8) [16]. Topical diclofenac has been shown to be efficacious in osteoarthritis, rheumatic arthritis, and soft tissue pain injuries such as sprains, ligament partial or total rupture, and contusions [16]. Oral diclofenac has been shown to have adverse vascular and gastrointestinal effects, and the efficacy of topical diclofenac is limited due to limited penetration through the skin tissue [15, 17, 18].

The ultrasound acoustic waves are generated by a piezoelectric element and require a dense medium to travel. Viscous ultrasound gel facilitates acoustic wave transmission from the transducer to the target tissue. The presence of the dense impedance-matched medium allows the transmission of acoustic vibration to the tissue with little or no loss of acoustic energy and intensity. In addition, the localized acoustic force can provide essential mechanical and thermal stimulation to increase profusion, porosity, and cellular activity [19, 20]; thereby synchronizing the mechano-transduction and diathermic properties of ultrasound along with topical NSAID application benefits. [19, 21, 22].

The United States Food and Drug Administration has cleared Sustained Acoustic Medicine (SAM) for treating musculoskeletal injuries. SAM provides low-intensity continuous ultrasound at 1.3 W and 0.132 W/cm², which equates to about 5,000 Joules per hour or just under 20,000 Joules for a typical 4-hour treatment session [10, 21, 23]. The meta-analysis by Winkler et al. demonstrated the clinical efficacy of the SAM in numerous musculoskeletal disorders, including myofascial pain, rotator cuff tendinopathy, knee osteoarthritis, and upper shoulder and neck pain [23]. In addition, Uddin et al. have elaborated on the underlying mechanism of LITUS and SAM in multiple pathologies such as traumatic bone fracture, osteoarthritis, thrombosis, and pain management [6].

The use of SAM with diclofenac ultrasound gel has been reported by Madzia et al. The SAM treatment with 1% topical diclofenac significantly alleviated the osteoarthritis-induced pain and improved Western Ontario McMaster Outcome (WOMAC) after 7 days of treatment [24]. The treatment efficacy and improved outcomes were attributed to the diathermic and mechanotransductive abilities of the SAM treatment over a 4-hour time. Jarit et al. presented the use of SAM with 2.5% diclofenac in the treatment of musculoskeletal injuries that failed to respond to physical therapy [30]. After 4-weeks of daily home-use patients reported a significant pain reduction, global health and functional improvement with SAM treatment.

There is a need for improved ultrasound coupling gels, patches, and devices for use in ultrasound therapies such as long duration ultrasound therapy.

The present disclosure is directed to overcoming these and other deficiencies in the art.

### SUMMARY OF THE INVENTION

The present disclosure generally relates to, inter alia, a high density ultrasound coupling gel, ultrasound coupling device (e.g., coupling gel patch), and ultrasound system for improved ultrasound coupling, thermal transfer, drug delivery, and long duration ultrasound treatment.

In one aspect, the present disclosure provides a high density ultrasound coupling gel that includes: (i) a water-based ultrasound transmission gel material in a concentration amount of between about 90 and 99 weight percent (wt %); and (ii) a sodium dopant in a concentration amount of between about 1 and 10 wt %. The water-soluble ultrasound transmission gel material is effective to conduct acoustic energy. The sodium dopant is effective to increase density of the water-based ultrasound transmission gel material. The high density ultrasound coupling gel has a density of between about 1 and 2 grams per milliliter (g/mL).

In another aspect, the present disclosure provides an ultrasound coupling device that includes: (i) a high density ultrasound coupling gel according to present disclosure; and (ii) a coupling compartment configured to hold the high density ultrasound coupling gel.

In another aspect, the present disclosure provides an ultrasound system that includes: (i) at least one ultrasound transducer; (ii) a power source operably connected to the ultrasound transducer; and (iii) a high density ultrasound coupling gel according to the present disclosure. The high density ultrasound coupling gel is effective for operably coupling to the at least one ultrasound transducer to apply therapeutic ultrasound energy to a subject.

In another aspect, the present disclosure provides a method of treatment involving: (i) providing an ultrasound system according to the present disclosure; and (ii) using the ultrasound system to apply ultrasound therapy to treat a subject in need of such therapy.

The high density ultrasound coupling gel of the present disclosure is a significant improvement over the existing art, including for using in drug delivery (e.g., delivery of an NSAID such as diclofenac) where the gel is formulated as a captured drug that may be activated when exposed to ultrasound. In contrast to existing ultrasound gels, the high density ultrasound coupling gel of the present disclosure has an increased density/acoustic impedance and heat transfer. Furthermore, the drug (e.g., an NSAID such as diclofenac) is uniformly distributed within the high density gel formulation. The high density ultrasound coupling gel of the present disclosure exhibits demonstrably improved ultrasound transfer and heat transfer away from the ultrasound producing transducer into the tissue. Moreover, the drug-deliver component is an additional benefit for use with the high density ultrasound coupling gel of the present disclosure. The high density ultrasound coupling gel of the present disclosure is configured to stay coupled for multiple hours, while existing gels are meant to be fully absorbed for delivery of therapeutic, which is a different purpose that the present disclosure. Thus, unlike existing ultrasound gels and systems used that include drug-filled gels for therapy or drug deliver, the high density ultrasound coupling gel of the present disclosure is not designed to be absorbed to deliver the drug. Instead, the high density ultrasound coupling gel of the present disclosure is designed and has the functionality of staying put between the transducer and the body and allowing long-term coupling (ultrasound and heat transfer), thereby resulting in a much smaller component focused on drug delivery over the longer-term time-course.

In another aspect, the present disclosure relates to the addition of a non-steroidal anti-inflammatory drugs (NSAID) or other agent into the coupling patch for improved acoustic impedance properties.

In another aspect, the present disclosure relates to the addition of a therapeutic agent into the coupling gel for improved ultrasound coupling and therapeutic action.

In another aspect, the present disclosure relates to the improved ultrasound coupling for deeper therapeutic delivery.

In certain aspects, the present disclosure relates to adding in a nonsteroidal anti-inflammatory drug into an ultrasound coupling gel in an ultrasound coupling patch to improve the acoustic impedance. By improving the acoustic impedance, the result is better ultrasound coupling and deeper penetration of ultrasound from an ultrasound coupling device, thereby improving its effectiveness. In certain embodiments, the present disclosure involves delivering topical drugs so as to provide a multi modal advantage.

In certain aspects, the present disclosure provides a drug/improved impedance matched gel for long duration ultrasound delivery.

In certain aspects, the present disclosure provides devices, systems, and methods for increasing the density of an ultrasound coupling gel by adding in pharmacotherapies to improve ultrasound coupling of energy from the device into the human body. This thereby allows the practitioner to treat deeper tissue. The added benefit is that the drug also provides pharmacokinetic advantage for reducing pain and inflammation as well.

In certain aspects, the present disclosure provides a patch gel having the following ingredients: aqueous ultrasound gel, diclofenac sodium and cellulose. The present disclosure also contemplates the use of any other NSAID, thickening agent, or ultrasound gel.

As provided herein, the present disclosure provides devices (including but not limited to ultrasound coupling gels, patches, and other devices), systems, and methods effective to provide a closer impedance match between the ultrasound gel and tissue.

As provided herein, the present disclosure provides devices, systems, and methods that use higher density gel so that the heating is better conducted from the losses in the transducer into the tissue as well.

In one aspect, the present disclosure provides an ultrasound coupling gel comprising: a gel component comprising a gel material effective to conduct acoustic energy; and a pharmacotherapeutic agent.

In one embodiment of the ultrasound coupling gel, the gel material is an aqueous ultrasound gel.

In one embodiment of the ultrasound coupling gel, the pharmacotherapeutic agent is a nonsteroidal anti-inflammatory drug (NSAID).

In one embodiment of the ultrasound coupling gel, the NSAID is selected from the group consisting of diclofenac, ibuprofen, naproxen, celecoxib, mefenamic acid, etoricoxib, indomethacin, and aspirin.

In one embodiment of the ultrasound coupling gel, the NSAID is diclofenac sodium.

In one embodiment of the ultrasound coupling gel, the ultrasound coupling gel further includes a thickening agent.

In one embodiment of the ultrasound coupling gel, the thickening agent is cellulose.

In one aspect, the present disclosure provides an ultrasound coupling device comprising an ultrasound coupling gel according to the present disclosure; and a coupling compartment configured to hold the ultrasound coupling gel.

In one embodiment of the ultrasound coupling device, the ultrasound coupling device is wearable.

In one embodiment of the ultrasound coupling device, the ultrasound coupling device is flexible.

In one aspect, the present disclosure provides an ultrasound system comprising: at least one ultrasound transducer; and at least one ultrasound coupling device of the present disclosure.

In one embodiment of the ultrasound system, the ultrasound coupling system is wearable.

In one embodiment of the ultrasound system, the ultrasound coupling system is flexible.

In one aspect, the present disclosure provides a method of treatment comprising: providing an ultrasound system of the present disclosure; and using the ultrasound system to apply ultrasound therapy a subject to treat the subject in need of said therapy.

In one embodiment of the method of the present disclosure, the ultrasound coupling system is wearable.

In one embodiment of the method of the present disclosure, the ultrasound coupling system is flexible.

In one embodiment of the method of the present disclosure, the ultrasound therapy is long duration ultrasound therapy.

In one embodiment of the method of the present disclosure, the ultrasound therapy is deep tissue ultrasound therapy.

### BRIEF DESCRIPTION OF THE DRAWINGS

For the purpose of illustrating aspects of the present invention, there are depicted in the drawings certain embodiments of the invention. However, the invention is not limited to the precise arrangements and instrumentalities of the embodiments depicted in the drawings. Further, if provided, like reference numerals contained in the drawings are meant to identify similar or identical elements.

FIGS. 1A-1B: Photographs of experimental set-up for intensity measurement. FIG. 1A: Without bovine muscle tissue. FIG. 1B: With bovine muscle tissue. The portions labeled as lowercase a, b, c, and d are as follows: a) hydrophone connected to oscilloscope, b) standard ultrasound coupling gel, c) SAM transducer, and d) bovine muscle tissue cut to be 1 cm thick.

FIGS. 2A-2B: Photographs of experimental set-up for deep tissue diathermy using bovine muscle tissue. FIG. 2A: The top (birds eye) view. FIG. 2B: The side view where the thermocouple probes were inserted. The portions labeled as lowercase a, b, c, d, e, f, and g are as follows: a) Bovine muscle tissue, b) SAM bandage containing ultrasound coupling gel, c) SAM transducer, d) OMEGA Thermocouple Probe, e) Thermocouple probe inserted at 1 cm depth, f) Thermocouple probe inserted at 2 cm depth, and g) Thermocouple probe inserted at 5 cm depth.

FIG. 3: PRISMA flow diagram for screening, enrollment, group assignment, and completion (100%) for participants in clinical study.

FIGS. 4A-4B: Photographs of human subject experimental set-up. FIG. 4A: Ultrasound device on the calf. FIG. 4B: Ultrasound device on the forearm. The portions labeled as lowercase a and b are as follows: a) Untreated skin surface micro thermocouple probe adhered in place with a bandage, and b) treated skin surface micro thermocouple probe placed directly underneath the active transducer and held in place with the SAM bandage (ultrasound patch).

FIG. 5: Graph illustrating intensity of SAM Ultrasound at different depths in bovine muscle tissue compared to the intensity threshold for therapeutic benefits of commercially available ultrasound treatment.

FIG. 6: Graph illustrating temperature during SAM stimulation on bovine muscle tissue at various depths (1 cm, 2 cm, 5 cm) using two different coupling patches (with and without 2.5% diclofenac). * (p>0.05) and ** (p<0.05).

FIGS. 7A-7B: Graphs illustrating average temperature values for human subject clinical study. FIG. 7A: Ultrasound device on the calf. FIG. 7B: Ultrasound device on the forearm. The data is mean temperature at each anatomic site is presented before and after 4 hour of SAM stimulation.*** (p<0.0001).

FIG. 8: Graph illustrating results from use of the ZetrOZ sonophoresis patch worn on medial and lateral knee for osteoarthritis (OA) pain treatment. Graph shows reduction in OA pain from baseline to study endpoint (bottom). Ultrasound 1.96-point decrease on NRS after 6-weeks over placebo (*p<0.04). Sonophoresis 3.43-point reductions in pain after 8 weeks (*p<0.001) over topical.

FIG. 9: Graph illustrating results from use of the ZetrOZ ultrasonic generator and diclofenac sonophoresis patch on bovine tissue model. Heating profile of sonophoresis patch at 1 cm, 2 cm and 5 cm tissue depth.

FIG. 10: Graph illustrating results from 8-week study of test subjects and changes in OA knee pain, stiffness, and improved function when using sonophoresis with high density gel with diclofenac compared with topical application of diclofenac. The graph shows WOMAC improvement for sonophoresis vs. topical (p=0.0127).

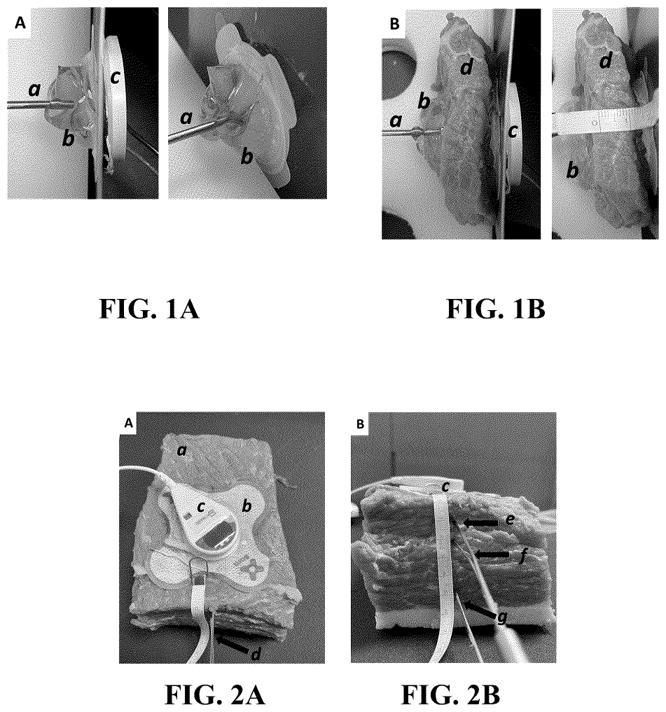

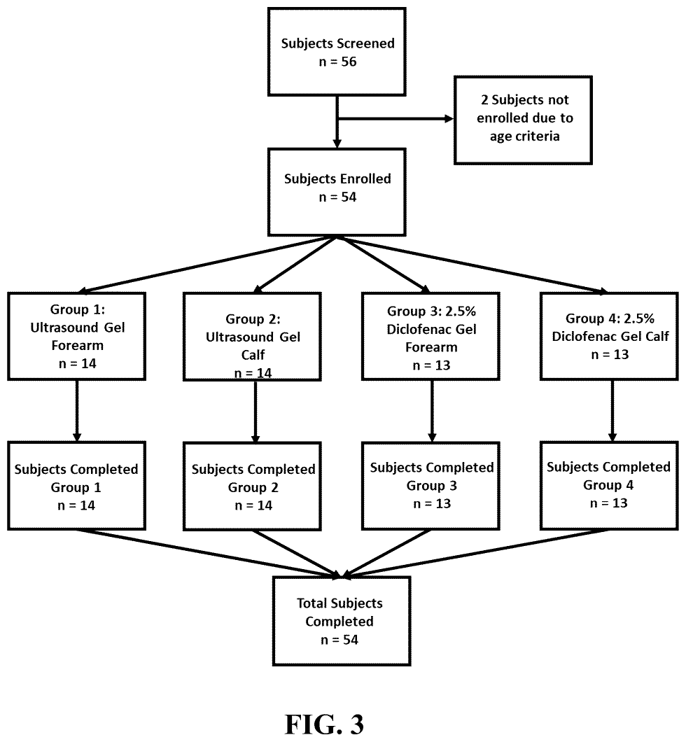

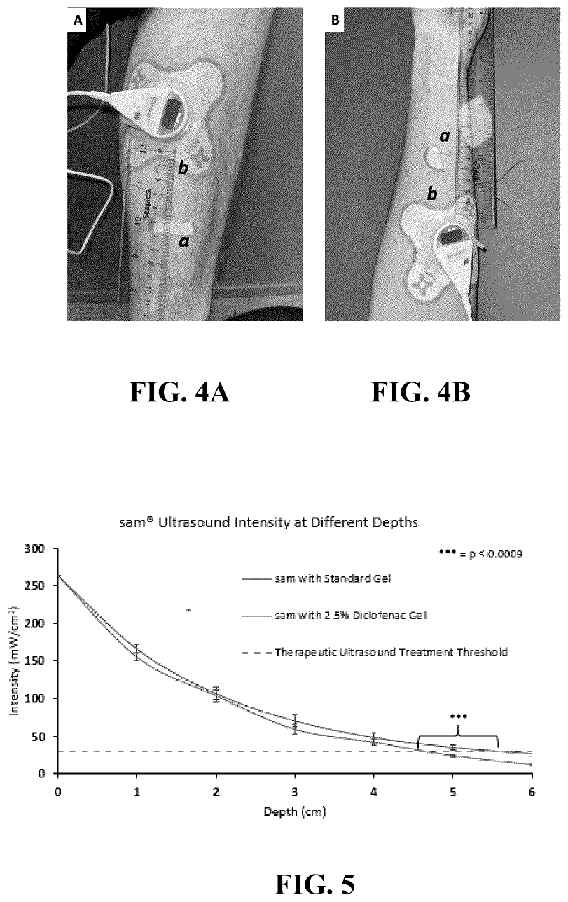

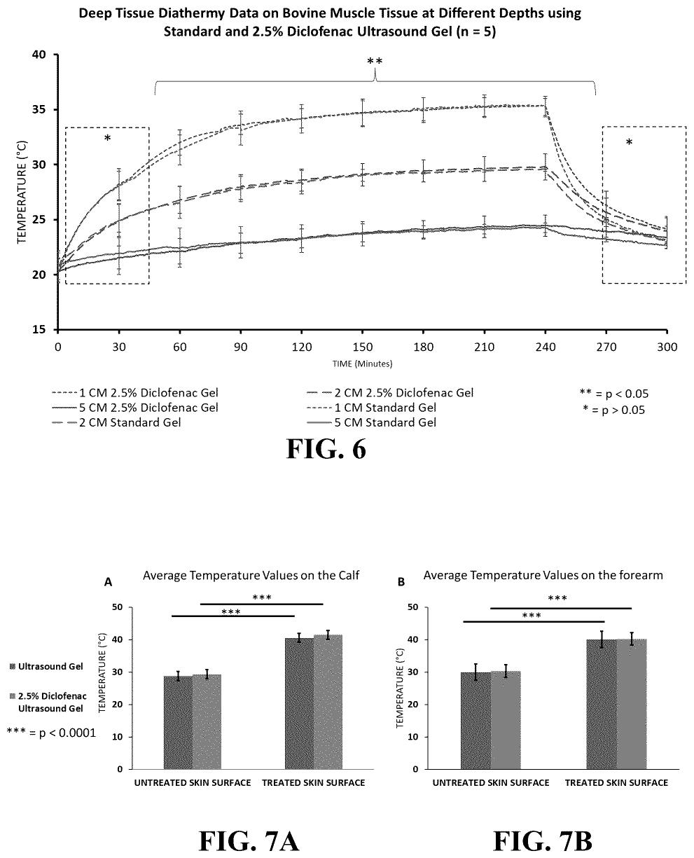

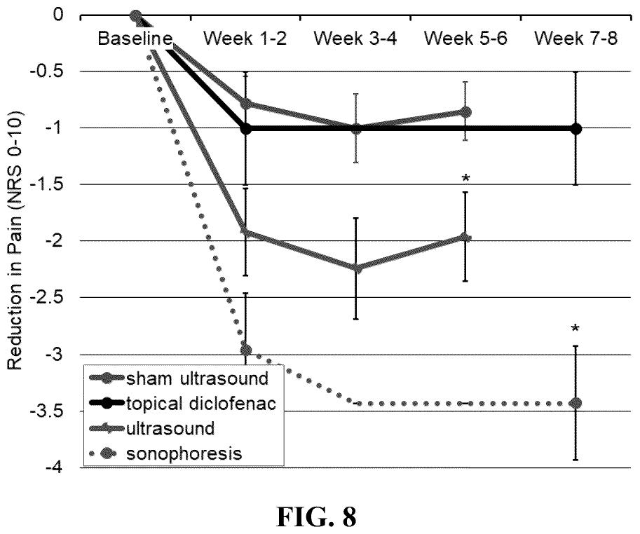

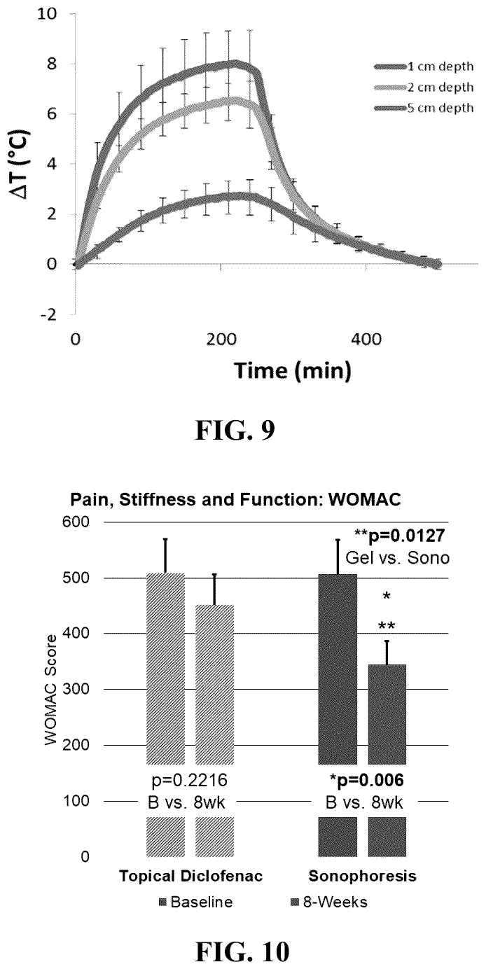

### DETAILED DESCRIPTION

The present disclosure generally relates to, inter alia, a high density ultrasound coupling gel, ultrasound coupling device (e.g., coupling gel patch), and ultrasound system for improved ultrasound coupling, thermal transfer, drug delivery, and long duration ultrasound treatment.

The high density ultrasound coupling gel, devices, systems, and methods of the present disclosure can be used with various ultrasound coupling patches, wearable ultrasound devices, and ultrasound therapeutic technology in the art. Samples of such complementary technology for use with the high density ultrasound coupling gel, devices, systems, and methods of the present disclosure include, without limitation, the following: WO2011/082402 (Ultrasound Coupling Device); WO2011/082407 (Portable Ultrasound System); WO2011/082408 (Low-Profile Ultrasound Transducer); WO2011/163570 (Hydrogel Ultrasound Coupling Device); WO2014/210063 (Low-Profile, Low-Frequency, and Low-Impedance Broad-Band Ultrasound Transducer and Methods Thereof); WO2014/210065 (Wearable Ultrasound Device); WO2015/130841 (Limited Use Ultrasonic Coupling Device); WO2018/102828 (Ultrasound Coupling Patch with Gel Capture Feature); and WO2020/046847 (Flexible and Wearable Long Duration Ultrasound Device), the disclosures of which are hereby incorporated by reference in their entirety.

Suitable ultrasound devices for use with the high density ultrasound coupling gel of the present disclosure can include any ultrasound device or ultrasound system that is configured or functional to apply therapeutic ultrasound to a mammalian subject (humans, animals, marine mammals). Suitable ultrasound devices and systems can include, without limitation, the Sustained Acoustic Medicine (SAM) ultrasound devices, ultrasound systems, and ultrasonic diathermy medical devices (ZetrOZ Systems LLC., Trumbull, CT).

In one aspect, the present disclosure relates to a high density ultrasound coupling gel that includes: (i) a water-based ultrasound transmission gel material in a concentration amount of between about 90 and 99 weight percent (wt %); and (ii) a sodium dopant in a concentration amount of between about 1 and 10 wt %. The water-soluble ultrasound transmission gel material is effective to conduct acoustic energy. The sodium dopant is effective to increase density of the water-based ultrasound transmission gel material. The high density ultrasound coupling gel has a density of between about 1 and 2 grams per milliliter (g/mL).

In certain embodiments, the high density ultrasound coupling gel has a speed of sound of between about 1450 and 1650 meters per second (m/s).

In certain embodiments, the high density ultrasound coupling gel has an acoustic impedance of between about 1.4 and 1.8 Megapascal-seconds per cubic meter (MPa-s/m³).

In certain embodiments, the high density ultrasound coupling gel has a pH of between about 6 and 8.

In certain embodiments, the high density ultrasound coupling gel has a viscosity of between about 100,000 and 400,000 centipoise (cP).

In certain embodiments, the high density ultrasound coupling gel has a speed of sound of between about 1450 and 1650 m/s, an acoustic impedance of between about 1.4 and 1.8 MPa-s/m³, a pH of between about 6 and 8, and a viscosity of between about 100,000 and 400,000 centipoise (cP).

In certain embodiments, the water-based ultrasound transmission gel material includes water at a concentration of between about 90 and 99 wt % and at least one component that includes, without limitation, a polyacrylic acid polymer, triethanolamine, monopropylene glycol, polyethylene oxide, and the like. Suitable water-based ultrasound transmission gel materials can include any water-based gel suitable for use for coupling an ultrasound transducer with a mammalian subject for transmitted ultrasound acoustic energy from the ultrasound transducer to the mammalian subject. Suitable water-based ultrasound transmission gel materials can include, without limitation, Aquasonic gels (GE HealthCare, Parker Labs), PolySonic gels (GE HealthCare), TheraSonic conductive gels (Compass Health Brands), and the like.

In certain embodiments, the sodium dopant includes sodium at a concentration that includes, without limitation, between about 0.18 and 1.5 wt %, between about 0.2 and 1.2 wt %, between about 0.4 and 1.0 wt %, and between about 0.6 and 0.8 wt %.

In certain embodiments, the sodium dopant includes a sodium compound that includes, without limitation, sodium chloride (NaCl), sodium sulphate (Na₂SO₄), sodium perchlorate (NaClO₄), sodium bromide (NaBr), sodium fluoride (NaF), sodium iodide (NaI), hypochlorite (NaClO), sodium bicarbonate (NaHCO₃), sodium hydroxide (NaOH), sodium gold thiosulfate (AuH₂Na₃O₆S₄⁺), and the like. Some non-sodium dopants can also be used to increase density, including, without limitation, selenious acid (H2SeO3), nitrous acid (HNO2), and the like.

When homogenously mixed into the water-based ultrasound transmission gel material, the sodium dopant, including sodium compounds and sodium itself, assists in increasing the density, acoustic impedance, and heat transfer of the gel. Therefore, compared to other water-based ultrasound gels, the high density ultrasound coupling gel has measurably better ultrasound transmission and coupling properties, particularly, but not limited to, the use as the ultrasound coupling gel for long-duration ultrasound therapeutic treatment. Furthermore, doping the gel with a drug (e.g., NSAID) is another added benefit of the high density ultrasound coupling gel of the present disclosure. For example, the high density ultrasound coupling gel combined into a patch has better heat transfer capability to allow the ultrasound system to perform better on patients (mammalian subjects in need of such therapy).

In certain embodiments, the sodium dopant includes a nonsteroidal anti-inflammatory drug (NSAID) and sodium.

In certain embodiments, the NSAID includes, without limitation, diclofenac, diclofenac sodium, diclofenac sodium with misoprostol, diclofenac potassium, flunixin meglumine, phenylbutazone, aspirin, acetaminophen, diflunisal, dipyrone, ketorolac, etodolac, tepoxalin, fenoprofen calcium, flurbiprofen, ibuprofen, indomethacin, celecoxib, ketoprofen, vedaprofen, meclofenamate sodium, mefenamic acid, tolfenamic acid, meloxicam, carprofen, nabumetone, naproxen, naproxen sodium, etoricoxib, piroxicam, tolmetin sodium, magnesium salicylate, choline salicylate, salsalate, sodium salicylate, alkyl salicylate, disalicylate, and the like.

In certain embodiments, the sodium dopant is present at a concentration amount that includes, without limitation, about 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, and 10 wt %.

In certain embodiments, the high density ultrasound coupling gel according to the present disclosure further includes a thickening agent at a concentration of between about 0.1 and 1.5 wt %. In certain embodiments, thickening agent can include, without limitation, cellulose, hydroxyethyl cellulose, and the like.

In certain embodiments, the high density ultrasound coupling gel according to the present disclosure further includes a preservative at a concentration of between about 0.01 and 1 wt %. In certain embodiments, the preservative can include, without limitation, methylparaben, propylparaben, diazolindinyl urea, sorbic acid, phenoxyethanol, ethylparaben, butylparaben, sodium butylparaben, caprylyl glycol, dehydroacetic acid, chlorphenesin, grape seed extract, and the like.

The high density ultrasound coupling gel according to the present disclosure can be made by combining the a water-based ultrasound transmission gel material with the sodium dopant using standard measurement and mixing techniques normally used in the relevant art for the same or similar compositions. In certain embodiments, the standard measurements and mixing techniques are followed so that the resulting high density ultrasound coupling gel of the present disclosure has a concentration amount of between about 90 and 99 wt % of the water-based ultrasound transmission gel material and a concentration amount of between about 1 and 10 wt % of the sodium dopant. Further, the standard measurements and mixing techniques are followed so that the resulting high density ultrasound coupling gel of the present disclosure is effective to conduct acoustic energy, so that the sodium dopant is effective to increase density of the water-based ultrasound transmission gel material, and/or so that the high density ultrasound coupling gel has a density of between about 1 and 2 grams per milliliter (g/mL).

In certain embodiments, the high density ultrasound coupling gel according to the present disclosure is made by combining all the ingredients into a rapid centrifugal speed mixer at an appropriate speed (RPM) for an appropriate amount of time to yield a high density ultrasound coupling gel having complete homogeneity. In a particular embodiment, the water-based ultrasound transmission gel material is Aquasonic gel (3.047 g), the sodium dopant is diclofenac sodium (0.079 g), and the thickener (also a stabilizer) is hydroxyethyl cellulose NF (0.043 g). These ingredients can be added together and mixed at 100× in a rapid centrifugal speed mixer for 10 minutes to yield a high density ultrasound coupling gel according to the present disclosure (3.17 g), with the high density ultrasound coupling having homogeneity of the ingredients.

In another aspect, the present disclosure relates to an ultrasound coupling device that includes: (i) a high density ultrasound coupling gel according to present disclosure; and (ii) a coupling compartment configured to hold the high density ultrasound coupling gel.

In one embodiment, the ultrasound coupling device is in the form of an ultrasound coupling patch containing the high density ultrasound coupling gel according to present disclosure. In accordance with the present disclosure, the use of the high density ultrasound coupling gel according to present disclosure in the patch results in improved acoustic coupling characteristics, as well as other improvements discussed herein.

In certain embodiments, the ultrasound coupling device is wearable.

In certain embodiments, the ultrasound coupling device is flexible.

In certain embodiments, the ultrasound coupling device is in the form of a gel patch and is made using measurements and techniques known to those of ordinary skill in the art. For example, the ultrasound coupling device can be manufactured by providing or producing the high density ultrasound coupling gel of the present disclosure and filling a compartment (patch) with the gel, and then sealing the compartment (patch). Various types of compartments and patch configurations are found in the art as referenced herein.

As provided herein, the ultrasound devices of the present disclosure can be manufactured and assembled using standard materials and techniques known in the art and/or taught herein, as long as the gel used to make the ultrasound system is a high density ultrasound coupling gel according to the present disclosure. To reiterate, the ultrasound device can include, without limitation, a SAM gel patch for use in a therapeutic ultrasound system such as SAM (ZetrOZ Systems, Trumbull, CT). See also WO2011/082402 (Ultrasound Coupling Device); WO2011/082407 (Portable Ultrasound System); WO2011/082408 (Low-Profile Ultrasound Transducer); WO2011/163570 (Hydrogel Ultrasound Coupling Device); WO2014/210063 (Low-Profile, Low-Frequency, and Low-Impedance Broad-Band Ultrasound Transducer and Methods Thereof); WO2014/210065 (Wearable Ultrasound Device); WO2015/130841 (Limited Use Ultrasonic Coupling Device); WO2018/102828 (Ultrasound Coupling Patch with Gel Capture Feature); and WO2020/046847 (Flexible and Wearable Long Duration Ultrasound Device), the disclosures of which are hereby incorporated by reference in their entirety.

In another aspect, the present disclosure relates to an ultrasound system that includes: (i) at least one ultrasound transducer; (ii) a power source operably connected to the ultrasound transducer; and (iii) a high density ultrasound coupling gel according to the present disclosure. The high density ultrasound coupling gel is effective for operably coupling to the at least one ultrasound transducer to apply therapeutic ultrasound energy to a subject.

As provided herein, the ultrasound system of the present disclosure can be manufactured and assembled using standard materials and techniques known in the art and/or taught herein, as long as the gel used with the ultrasound system is a high density ultrasound coupling gel according to the present disclosure. To reiterate, the ultrasound system can include, without limitation, a SAM ultrasound system (ZetrOZ Systems, Trumbull, CT). See also WO2011/082402 (Ultrasound Coupling Device); WO2011/082407 (Portable Ultrasound System); WO2011/082408 (Low-Profile Ultrasound Transducer); WO2011/163570 (Hydrogel Ultrasound Coupling Device); WO2014/210063 (Low-Profile, Low-Frequency, and Low-Impedance Broad-Band Ultrasound Transducer and Methods Thereof); WO2014/210065 (Wearable Ultrasound Device); WO2015/130841 (Limited Use Ultrasonic Coupling Device); WO2018/102828 (Ultrasound Coupling Patch with Gel Capture Feature); and WO2020/046847 (Flexible and Wearable Long Duration Ultrasound Device), the disclosures of which are hereby incorporated by reference in their entirety.

In another aspect, the present disclosure relates to a method of treatment involving: (i) providing an ultrasound system according to the present disclosure; and (ii) using the ultrasound system to apply ultrasound therapy to treat a subject in need of such therapy.

In certain embodiments of the method of treatment of the present disclosure, the ultrasound therapy is long duration ultrasound therapy of up to about 12 hours.

In certain embodiments of the method of treatment of the present disclosure, the ultrasound therapy is deep tissue ultrasound therapy.

In certain embodiments of the method of treatment of the present disclosure, the deep tissue ultrasound therapy delivers therapeutic acoustic energy to a depth of up to about 10 centimeters (cm) into the tissue of the subject.

In certain embodiments of the method of treatment of the present disclosure, the high density ultrasound coupling gel provided with the ultrasound system is effective to increase ultrasound coupling between the ultrasound transducer and the subject's tissue.

In certain embodiments of the method of treatment of the present disclosure, the high density ultrasound coupling gel provided with the ultrasound system has an increased heat capacity effective to facilitate and maintain heat transfer from the ultrasound transducer to the subject's tissue to improve long duration ultrasound therapy.

In certain embodiments of the method of treatment of the present disclosure, the ultrasound therapy includes delivering an NSAID to the subject's tissue to improve treatment.

In certain embodiments of the method of treatment of the present disclosure, the treatment is for conditions including, without limitation, a musculoskeletal condition, soft tissue injury, pain relief, inflammation, osteoarthritis, rhematic arthritis, contusion, ligament injury, muscle spasm, tendinopathy, bone fracture healing, and the like.

In certain embodiments of the method of treatment of the present disclosure, the high density ultrasound coupling gel is effective to improve pain relief to the subject compared to treatment with a lower density ultrasound coupling gel.

In certain embodiments of the method of treatment of the present disclosure, the high density ultrasound coupling gel is effective to improve healing of to the subject compared to treatment with a lower density ultrasound coupling gel.

In certain embodiments of the method of treatment of the present disclosure, the high density ultrasound coupling gel is effective to increase ultrasound coupling efficacy during the treatment compared to treatment with a lower density ultrasound coupling gel.

In certain embodiments of the method of treatment of the present disclosure, the high density ultrasound coupling gel is effective to reduce excess heating or other pain sensations by the subject during time of treatment compared to treatment with a lower density ultrasound coupling gel for the same amount of time of treatment.

In certain embodiments of the method of treatment of the present disclosure, the high density ultrasound coupling gel is effective to increase length of time and quality of ultrasound coupling during the treatment compared to treatment with a lower density ultrasound coupling gel.

The methods of treatment of the present disclosure can be used in accordance with any long-duration ultrasound therapeutic treatment protocols, devices, and systems, including, without limitation, in low-intensity continuous ultrasound (LICUS), Sustained Acoustic Medicine (SAM), long-duration low intensity therapeutic ultrasound (LITUS) treatment protocols, devices, and systems.

### EXAMPLES

The following examples are intended to illustrate particular embodiments of the present disclosure, but are by no means intended to limit the scope of the present disclosure.

What is claimed is:

1. A high density ultrasound coupling gel comprising:
a water-based ultrasound transmission gel material in a concentration amount of between about 90 and 99 weight percent (wt %), said water-soluble ultrasound transmission gel material being effective to conduct acoustic energy; and
a sodium dopant in a concentration amount of between about 1 and 10 wt %, said sodium dopant being effective to increase density of the water-based ultrasound transmission gel material,
wherein the high density ultrasound coupling gel has a density of between about 1 and 2 grams per milliliter (g/mL).

2. The high density ultrasound coupling gel according to claim 1, wherein the high density ultrasound coupling gel has a speed of sound of between about 1450 and 1650 meters per second (m/s).

3. The high density ultrasound coupling gel according to claim 1, wherein the high density ultrasound coupling gel has an acoustic impedance of between about 1.4 and 1.8 Megapascal-seconds per cubic meter (MPa-s/m³).

4. The high density ultrasound coupling gel according to claim 1, wherein the high density ultrasound coupling gel has a pH of between about 6 and 8.

5. The high density ultrasound coupling gel according to claim 1, wherein the high density ultrasound coupling gel has a viscosity of between about 100,000 and 400,000 centipoise (cP).

6. The high density ultrasound coupling gel according to claim 1, wherein the high density ultrasound coupling gel has a speed of sound of between about 1450 and 1650 m/s, an acoustic impedance of between about 1.4 and 1.8 MPa-s/m³, a pH of between about 6 and 8, and a viscosity of between about 100,000 and 400,000 centipoise (cP).

7. The high density ultrasound coupling gel according to claim 1, wherein the water-based ultrasound transmission gel material comprises water at a concentration of between about 90 and 99 wt % and at least one component selected from the group consisting of a polyacrylic acid polymer, triethanolamine, monopropylene glycol, and polyethylene oxide.

8. The high density ultrasound coupling gel according to claim 1, wherein the sodium dopant comprises sodium at a concentration selected from the group consisting of between about 0.18 and 1.5 wt %, between about 0.2 and 1.2 wt %, between about 0.4 and 1.0 wt %, and between about 0.6 and 0.8 wt %.

9. The high density ultrasound coupling gel according to claim 1, wherein the sodium dopant comprises a sodium compound selected from the group consisting of sodium chloride (NaCl), sodium sulphate (Na₂SO₄), sodium perchlorate (NaClO₄), sodium bromide (NaBr), sodium fluoride (NaF), sodium iodide (NaI), hypochlorite (NaClO), sodium bicarbonate (NaHCO₃), sodium hydroxide (NaOH), and sodium gold thiosulfate (AuH₂Na₃O₆S₄⁺).

10. The high density ultrasound coupling gel according to claim 1, wherein the sodium dopant comprises a nonsteroidal anti-inflammatory drug (NSAID) and sodium.

11. The high density ultrasound coupling gel according to claim 10, wherein the NSAID is selected from the group consisting of diclofenac, diclofenac sodium, diclofenac sodium with misoprostol, diclofenac potassium, flunixin meglumine, phenylbutazone, aspirin, acetaminophen, diflunisal, dipyrone, ketorolac, etodolac, tepoxalin, fenoprofen calcium, flurbiprofen, ibuprofen, indomethacin, celecoxib, ketoprofen, vedaprofen, meclofenamate sodium, mefenamic acid, tolfenamic acid, meloxicam, carprofen, nabumetone, naproxen, naproxen sodium, etoricoxib, piroxicam, tolmetin sodium, magnesium salicylate, choline salicylate, salsalate, sodium salicylate, alkyl salicylate, and disalicylate.

12. The high density ultrasound coupling gel according to claim 1, wherein the sodium dopant is present at a concentration amount selected from the group consisting of about 1 wt %, 1.5 wt %, 2 wt %, 2.5 wt %, 3 wt %, 3.5 wt %, 4 wt %, 4.5 wt %, 5 wt %, 5.5 wt %, 6 wt %, 6.5 wt %, 7 wt %, 7.5 wt %, 8 wt %, 8.5 wt %, 9 wt %, 9.5 wt %, and 10 wt %.

13. The high density ultrasound coupling gel according to claim 1, further comprising a thickening agent at a concentration of between about 0.1 and 1.5 wt % and selected from the group consisting of cellulose and hydroxyethyl cellulose.

14. The high density ultrasound coupling gel according to claim 1, further comprising a preservative at a concentration of between about 0.01 and 1 wt % and selected from the group consisting of methylparaben, propylparaben, diazolindinyl urea, sorbic acid, phenoxyethanol, ethylparaben, butylparaben, sodium butylparaben, caprylyl glycol, dehydroacetic acid, chlorphenesin, and grape seed extract.

15. An ultrasound coupling device comprising:
the high density ultrasound coupling gel according to claim 1; and
a coupling compartment configured to hold the high density ultrasound coupling gel.

16. The ultrasound coupling device according to claim 15, wherein the ultrasound coupling device is wearable.

17. The ultrasound coupling device according to claim 15, wherein said ultrasound coupling device is flexible.

18. An ultrasound system comprising:
at least one ultrasound transducer;
a power source operably connected to the ultrasound transducer; and
the high density ultrasound coupling gel according to claim 1, wherein the high density ultrasound coupling gel is effective for operably coupling to the at least one ultrasound transducer to apply therapeutic ultrasound energy to a subject.

19. A method of treatment comprising:
providing an ultrasound system according to claim 18; and
using the ultrasound system to apply ultrasound therapy to treat a subject in need of said therapy.

20. The method according to claim 19, wherein the ultrasound therapy is long duration ultrasound therapy of up to about 12 hours.

21. The method according to claim 19, wherein the ultrasound therapy is deep tissue ultrasound therapy.

22. The method according to claim 21, wherein the deep tissue ultrasound therapy delivers therapeutic acoustic energy to a depth of up to about 10 centimeters (cm) into the tissue of the subject.

23. The method according to claim 19, wherein the high density ultrasound coupling gel provided with the ultrasound system is effective to increase ultrasound coupling between the ultrasound transducer and the subject's tissue.

24. The method according to claim 19, wherein the high density ultrasound coupling gel provided with the ultrasound system has an increased heat capacity effective to facilitate and maintain heat transfer from the ultrasound transducer to the subject's tissue to improve long duration ultrasound therapy.

25. The method according to claim 19, wherein the ultrasound therapy includes delivering an NSAID to the subject's tissue to improve treatment.

26. The method according to claim 19, wherein the treatment is for conditions selected from the group consisting of a musculoskeletal condition, soft tissue injury, pain relief, inflammation, osteoarthritis, rhematic arthritis, contusion, ligament injury, muscle spasm, tendinopathy, and bone fracture healing.

27. The method according to claim 19, wherein the high density ultrasound coupling gel is effective to improve pain relief to the subject compared to treatment with a lower density ultrasound coupling gel.

28. The method according to claim 19, wherein the high density ultrasound coupling gel is effective to improve healing of to the subject compared to treatment with a lower density ultrasound coupling gel.

29. The method according to claim 19, wherein the high density ultrasound coupling gel is effective to increase ultrasound coupling efficacy during the treatment compared to treatment with a lower density ultrasound coupling gel.

30. The method according to claim 19, wherein the high density ultrasound coupling gel is effective to reduce excess heating or other pain sensations by the subject during time of treatment compared to treatment with a lower density ultrasound coupling gel for the same amount of time of treatment.

31. The method according to claim 19, wherein the high density ultrasound coupling gel is effective to increase length of time and quality of ultrasound coupling during the treatment compared to treatment with a lower density ultrasound coupling gel.
